(19) United States
(12) Patent Application Publication
(10) Pub. No.: US 2011/0015500 A1
(43) Pub. Date: Jan. 20, 2011

(54) METHOD FOR DETERMINING CUSTOMIZED ESSENTIAL OIL BLEND

(75) Inventor: MON-HAN WU, TAINAN (TW)

Correspondence Address: J C PATENTS, 4 VENTURE, SUITE 250, IRVINE, CA, 92618, US

(73) Assignee: SUSTINEO BIOTECHNOLOGY CO., LTD., TAINAN (TW)

(21) Appl. No.: 12/838,281

(22) Filed: Jul. 16, 2010

Related U.S. Application Data
(60) Provisional application No. 61/226,351, filed on Jul. 17, 2009.

(51) Int. Cl. A61B 5/00; A61B 5/02; A61B 5/0402; A61B 5/0295; A61B 5/026

(52) U.S. Cl. 600/301; 600/481; 600/509; 600/323; 600/528

(57) ABSTRACT

A scientific method and system for determining a customized essential oil blend through accessing a physiolocial data from a user are disclosed in the present invention. The physiological data is heart rate vairiability (HRV), which is associated with cardiac diseases and the autonomic nervous system.

### CROSS REFERENCE TO RELATED APPLICATION

This application claims priority to U.S. Provisional application Ser. No. 61/226,351 filed on Jul. 17, 2009. All disclosure of the provisional application is incorporated herein by reference.

### FIELD OF THE INVENTION

The present invention is directed to a method and a system for determining essential oil, and more particularly, to a method and a system for determining a customized essential oil blend.

### BACKGROUND OF THE INVENTION

Aromatherapy is the practice of using volatile plant oils, including essential oils, for psychological and physical well-being. Commonly, aromatherapy is practiced as a type of alternative medicine. Essential oils are known to be highly concentrated substances extracted from flowers, leaves, stalks, fruits, or roots, and may also distilled from resins, and can be blended for a specific therapeutic application. However, some oils can cause allergic reactions or hypersensitization in some users. Through interrogative enquiry, aromatherapists can avoid the abovementioned situation and formulate personalized essential oils according to empirical rules. Consequently, how customized essential oil blends are formulated for different people at different times is very subjective and lacks corroborative scientific evidences.

Heart rate variability (HRV) is the measurements of interval variations during heartbeats. Heart rate is modulated by breath, blood pressure, the endocrine system, emotions, and the autonomic nervous system (ANS). Alterations in HRV have been reported to be associated with various pathologic conditions, such as hypertension, hemorrhagic shock, and septic shock. Also, HRV can also indicate the emotional state of a user.

Previous research has reported that applying essential oil would alter physiological indexes, such as blood pressure, heart rate, HRV, sympathetic nervous activity and parasympathetic nervous activity. Emotion states and the modulation of the ANS of a user was evaluated through analyzing HRV parameters after using essential oil.

U.S. Pub. No. 2002/0081341 reveals a method for formulating an essential oil-based composition. The essential oil blend was determined by analyzing personality and preference. U.S. Pub. No. 2006/0142968 discloses a home control system, which aims for optimum sleeping environments by controlling home facilities according to galvanic skin responses and heart rate variability. However, the disclosure only focuses on optimizing sleeping environments and does not teach how to choose essential oil (blends) per database calculation. The prior studies analyzed the distinguishing properties of essential oils via observing HRV parameters at resting state and after applying the essential oils. However, a systematic HRV response database of essential oils is still lacking.

### BRIEF DESCRIPTION OF THE DRAWINGS

FIG. 1 describes the overview of the method for customized essential oil blend formulation.

FIG. 2 describes the essential oil set modification process.

FIG. 3 describes the database processing algorithm which determines the appropriate essential oil set according to HRV response.

### DETAILED DESCRIPTION OF THE INVENTION

The present invention describes a method for determining a customized essential oil blend for use in, but not limited to, aromatherapy. The method comprises the steps of: measuring the heart rate variability (HRV) of a user; and generating a formulation of a customized essential oil blend according to an HRV-essential oil database and the HRV measurement of the user.

The customized essential oil blend mentioned above is composed of at least one pure essential oil and/or at least one commercial essential blend collection. In an embodiment of the invention, the customized essential oil blend can be chosen from the essential oil set of the HRV-essential oil database. The essential oil set comprises, but is not limited to, Ajowan, Amber, Amyris, Angelica, Basil, Bay, Benzoin Tincture, Bergamot, Black Pepper, Buchu, Cade, Cajeput, Camphor, Caraway, Cardamom, Carrot Seed, Cassia, Catnip, Cedarwood Oils, Celery Seed, Chamomile, Cinnamon, Citronella, Clary Sage, Clove Bud, Copibia, Coriander, Cubeb, Cumin Seed, Black Cumin Seed, Cypress, Davana, Dill Seed, Elemi, Eucalyptus, Fennel, Fir Needle, Frankincense Oils, Galangal, Galbanum, Geranium Oils, Ginger, Gingergrass, Grapefruit, Ho Leaf, Hyssop, Inmortelle, Juniper, Kanuka, Labdanum, Laurel, Lavandin, Lavender, Lemon, Lemon Verbena, Lemongrass, Lime, Linaloe Wood, Litsea Cubeba, Lovage, Mandarin, Manuka, Marjoram, Myrrh, Myrtle, Neroli, Niaouli, Nutmeg, Orange, Origanum, Palmarosa, Parsley, Patchouli, Peppermint, Petitgrain, Pimento, Pine, Ravensara, Rose Otto, Rosemary, Rosewood, Sage Spanish, Sandalwood, Savoury, Silver Fir Needle, Spearmint, Spikenard, Star Anise, Tagetes, Tangerine, Tarragon, Tea Tree, Thyme, Turmeric, Valerian Root, Vetivert, White Birch, Yarrow, Ylang Ylang, Ambrette Seed, Hibiscus Abelmoschus, Broom, Calendula, Carnation, Cassie, Champaca, Elder, Everlasting, Frangipani, Hyacinth, Jasmine, Linden Blossom, Narcissus, Oakmoss, Osmanthus, Rose Abs, Tonka Bean, Tuberose, Vanilla, Violet Leaf.

The method can further comprise a step of inputting a personal profile. The personal profile can be separated into three parts: subjective feeling about essential oils, personal information, and physiological conditions. In an embodiment of the invention, the subjective feeling about essential oils includes preference for and against certain essential oils. The personal information used herein includes, but is not limited to, age, gender, and/or race. The physiological conditions comprise personal medical history, pregnancy, breast feeding, length of sleep, and/or information regarding allergic essential oil(s). Also, a user may fill out questionnaires to evaluate his or her emotional state.

The purpose of inputting the personal profile is because although a user can clearly define which essential oil(s) they dislike and are allergic to; he or she may not have adequate knowledge of the properties of each essential oil. Through inputting physiological conditions, one could preliminary exclude or include some of the essential oils. For example, a user with high blood pressure should avoid Eucalyptus, Rosemary, and Thyme; a pregnant woman should refrain from Lavender, Basil, Sage, and Rosemary.

HRV, the variation of time intervals between heartbeats (also known as RR intervals or NN intervals), can be measured by electrocardiography (ECG), photoplethysmograph (PPG) or by a microphone in the present invention. The mentioned microphone is used to detect the sound waves of pulses. HRV could be analyzed by time domain methods, frequency domain methods and non-linear methods. These methods are used for calculating a series of parameters, which could refer to dissimilar physiological significances.

The HRV parameters used herein include, but are not limited to, power in high frequency range (HF, 0.15-0.4 Hz), power in low frequency range (LF, 0.04-0.15 Hz), power in very low frequency range (VLF, <0.04 Hz), total power (TP), LF/HF, nLF (normalized LF), nHF (normalized HF), stress index (SI), the standard deviation of the NN interval (SDNN), and the square root of the mean of the sum of the squares of differences between adjacent NN intervals (RMSSD). Table 1 shows the normal range and warning range of each HRV parameter.

TABLE 1
Overview of the normal range and warning range of HRV measurements

| Measurement parameter | Normal range | Warning range |
|---|---|---|
| HR (beat/min) | 60-100 | >100 or <60 |
| SDNN(ms) | >30 | <30 |
| RMSSD(ms) | >20 | <20 |
| nHF(nu) | 30-55 | >80 or <20 |
| nLF(nu) | 45-70 | >80 or <20 |
| LF/HF | 1.5-2.0 | >2.0 or <1.5 |
| PSI | 20-50 | >50 |
| TP(ms²) | 1000-2000 | <1000 |

HR: heart rate
SDNN: standard deviation of all NN intervals
RMSSD: the square root of the mean of the sum of the squares of differences between adjacent NN intervals
nu: normalized units
HF: high frequency
LF: low frequency
TP: total power
PSI: physiological strain index

The HRV-essential oil database used herein is built according to the changes of HRV parameters in response to a series of essential oils. The HRV-essential oil database could be built in accordance with experimental results and/or prior research. For example, Peng SM taught that the percentage change of LF/HF of participants increased after inhaling Citrus bergamia essential oil (Peng SM, et al., Effects of music and essential oil inhalation on cardiac autonomic balance in healthy individuals, The Journal of Alternative and Complementary Medicine. January 2009, 15(1): 53-57). Collecting the changes of HRV parameters from an examiner before and after applying different essential oils may be another way to construct the HRV-essential oil database. An essential oil may be applied through rubbing it in the skin or inhalation through nasal cavity.

The present invention also provides a system for determining a customized essential oil blend. The system comprises a physiological signal monitor, which accesses a physiological signal of user; an HRV-essential oil database; and a data processing device for determining the customized essential oil blend. In the embodiment, the physiological signal is HRV, ECG, electromyography (EMG), electrooculography (EOG), electroencephalography (EEG) or pulse wave velocity (PWV). In an embodiment, the physiological signal is HRV, which is detected by an HRV monitor. The HRV monitor could be electrocardiography monitor, photoplethysmograph monitor, or acoustic blood flow detector, which uses a microphone to detect pulses.

The data processing device of the system is a computer, a notebook computer, a mobile phone, a console, a personal digital assistant or another data-analyzing device. The data processing device also comprises a database processing algorithm, which is used to analyze signals accessed from the HRV monitor, which collects the physiological signals and calculates parameters including HF, LF, VLF, LF/HF, TP, SI, SDNN, and RMSSD, and determines the customized essential oil blend based on the HRV parameters, a personal profile and the HRV-essential oil database.

The data processing device is further equipped with the essential oils accossiated with special physiological states. The special physiological states include, but are not limited to, hypertension, hypotension, diabetes.

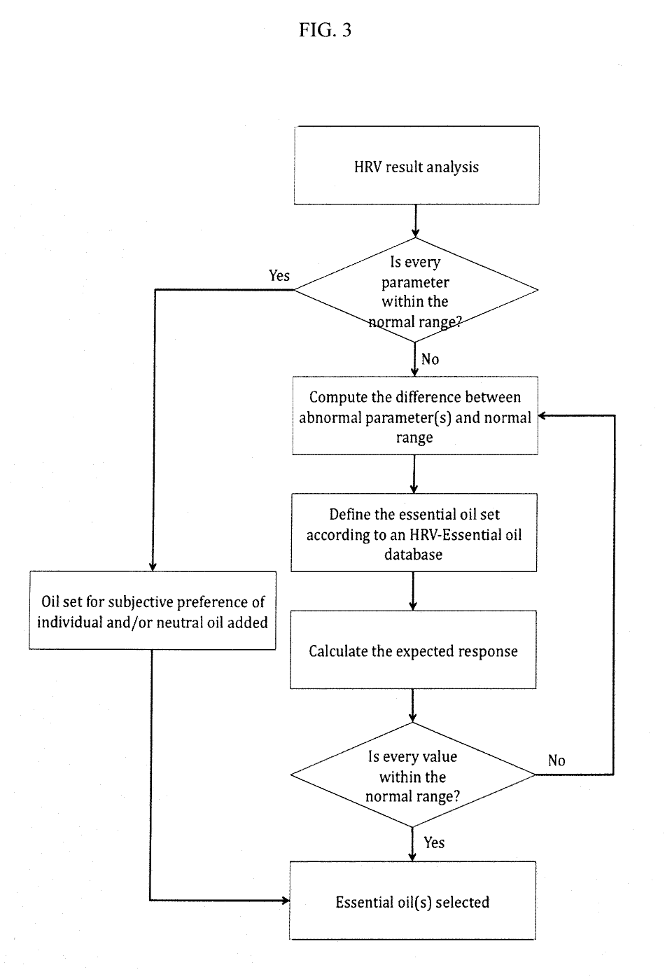

After measuring the HRV parameters of the user, the system determines the customized essential oil blend through a database evaluation process as FIG. 3 describes. The data processing device decides whether the HRV parameters of the user fall within the normal range or not. If only one of the HRV parameters is out of range, the data processing device accesses the appropriate essential oil set from HRV-essential oil database and then calculates the expected response to make sure that the HRV parameter will be improved after using the chosen essential oil set. If every HRV parameter of the user is within the normal range, the data processing device would choose the essential oil based on the user's preference.

The system further comprises a display to present the final calculation result to the user. The aromatherapist, if a tyro at aromatherapy, can objectively formulate the essential oil blend according to the result of the present system and apply the blend to the customer. Therefore, the present invention provides a scientific way for the aromatherapist and the general user who lack professional knowledge to define the most appropriate essential oil blend at once.

### EXAMPLE

### Example 1

Building an HRV-Essential Oil Database

The changes in HRV parameters in response to different essential oils were built in the database in the present invention. The HRV of individuals were recorded at a resting state and after applying the essential oil.

Two essential oils, Ylang Ylang (pure oil from ARITISCENT®) and Quiet Night (a commercial oil blend from THE BODY SHOP®), were applied to at least 10 health participants for each essential oil, and the results were examined. The HRV of the participants was measured by an ECG detector. All experiments were performed in a closed and air-conditioned room. Only one essential oil was applied each time in the room. The room was ventilated with fresh air for at least 2 hours between each experiment. The participants filled out questionnaires about their emotional state, physical condition, medical history, allergic essential oil(s), and preference of essential oil(s), before the experiment. All participants maintained normal breath, were seated, and were not allowed to talk, drink, or eat during the experiment. After a 10-minute rest, the HRV of the participants were measured as the rest state data, and then the participants inhaled a fixed amount of an essential oil which was diffused by an aroma diffuser for 10 minutes.

As the results in table 2 demonstrate, Ylang Ylang caused significant increases in HF and LF, while Quiet night only caused a remarkable increase in LF. By repeating this experiment through applying different essential oils, an HRV-essential oil database was built.

TABLE 2
The changes of HRV parameters after applying essential oil

| | | | ΔLF | ΔHF | |
|---|---|---|---|---|---|
| | ΔHR (%) | ΔSDNN (%) | (%) | (%) | ΔLF/HF (%) |
| Ylang Ylang | −4.96 | 17.01 | 78.98 | 71.06 | 40.61 |
| Quiet Night | −3.04 | 27.59 | 70.17 | 18.87 | 38.04 |

### Example 2

Generating a Customized Essential Oil Blend

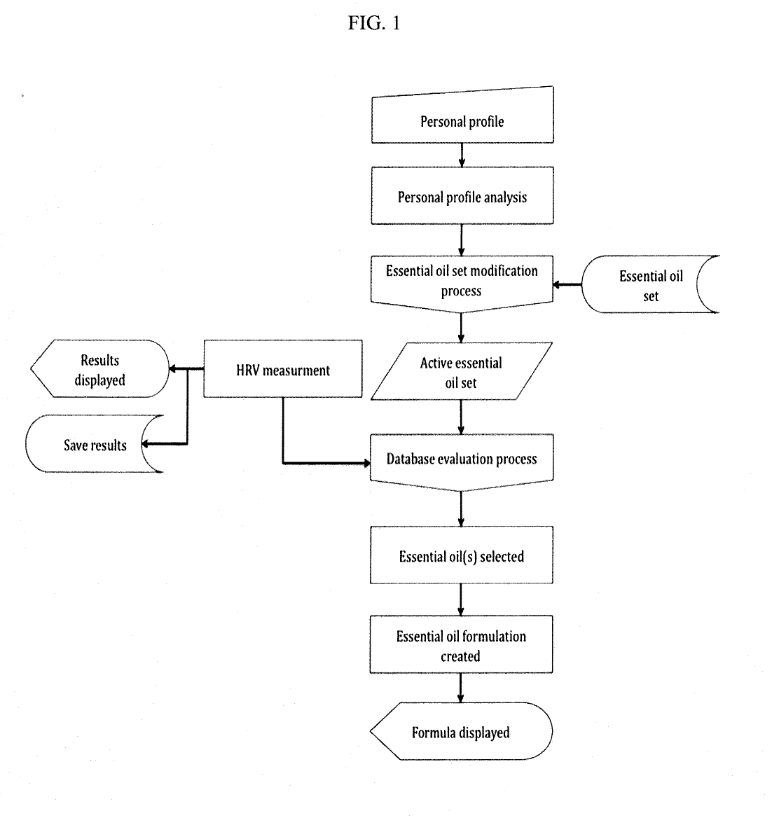

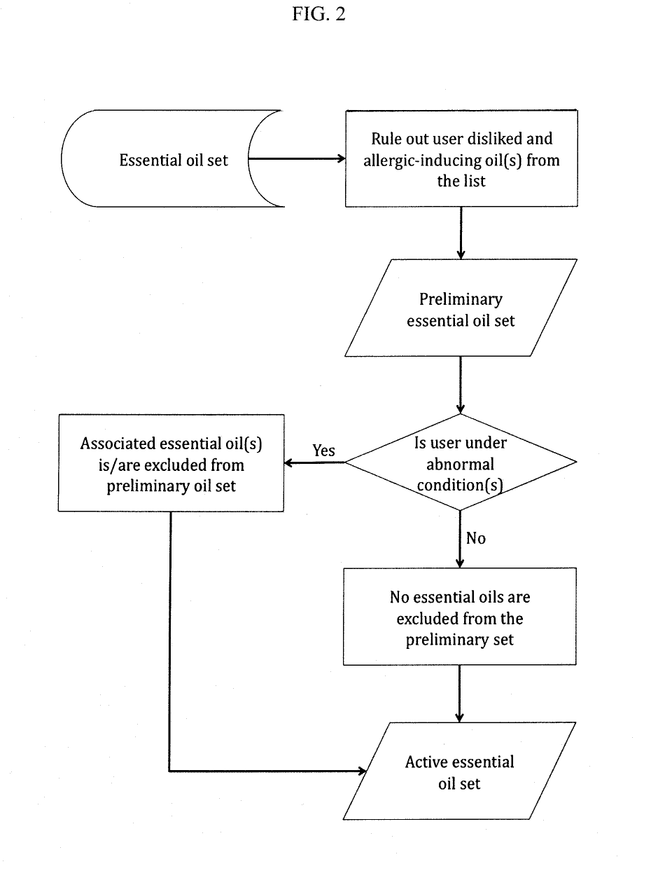

The overview of determining a customized essential oil blend is shown in FIG. 1. First, a personal profile is entered into the system. The profile may include preferences, medical history, allergens, illnesses, physical and emotional conditions and objectives of the user. After analyzing the personal profile, some essential oils which the user dislikes or are allergic to may be preliminarily ruled out from the essential oil set through an essential oil universal modification process (shown in FIG. 2). If the user reportes that he or she is under an abnormal physiological condition in his or her personal profile, the associated essential oil(s) may be excluded from the preliminary oil set. Said abnormal physiological condition could be pregnancy, breast feeding, hypertension, diabetes, insomnia, sleep apnea, cardiac diseases. For example, lavender, basil, sage, and rosemary should not be used during pregnancy. The essential oils remaining after the modification process constitute an active essential oil set.

The HRV parameters of the user are measured and entered into the system. If at least one of the HRV parameters of the user was out of normal range, the system evaluates what percentage of the parameters are out of range, selects essential oil(s) from the above active essential oil set, and calculates an appropriate essential oil formula to ameliorate the physical and psychological conditions of the user. If all HRV parameters of the user are within the normal range, the system will calculate the appropriate essential oil formula per the subjective preference of the user or choose a neutral essential oil that would not alter the HRV parameters.

1. A method for determining a customized essential oil blend, comprising
(a) measuring a heart rate variability (HRV) of a user; and
(b) generating a formulation of a customized essential oil blend according to an HRV-essential oil database and the HRV of the user.

2. The method of claim 1, which further comprises a step of inputting a personal profile.

3. The method of claim 2, wherein the personal profile comprises at least one of subjective feeling about essential oils, personal information, and physiological condition.

4. The method of claim 3, wherein the physiological condition comprises at least one of medical history, an allergic essential oil, and sleep time.

5. The method of claim 3, wherein the personal information comprises at least one of age, gender, and race.

6. The method of claim 1, wherein the HRV is measured by electrocardiography, photoplethysmograph, or by using a microphone.

7. The method of claim 1, wherein the heart rate variability comprises at least one of high frequency (HF), low frequency (LF), very low frequency (VLF), low frequency/high frequency (LF/HF), total power (TP), stress index (SI), standard deviation of the NN interval (SDNN), and the square root of the mean of the sum of the squares of differences between adjacent NN intervals (RMSSD).

8. The method of claim 1, wherein the customized essential oil blend comprises at least one of a pure essential oil and a commercial essential oil blend collection.

9. A system for determining a customized essential oil blend, comprising
(a) a heart rate variability (HRV) monitor, which accesses HRV parameters of a user;
(b) an HRV-essential oil database; and
(c) a data processing device for determining the customized essential oil blend.

10. The system of claim 9, wherein the heart rate variability monitor is a electrocardiography monitor, a photoplethysmograph monitor, or an acoustic blood flow detector.

11. The system of claim 9, wherein the data processing device is used for analyzing signals accessed by the HRV monitor.

12. The system of claim 9, wherein physiological signals of the user are the HRV parameters.

13. The system of claim 9, wherein the HRV parameters comprises at least one of high frequency (HF), low frequency (LF), very low frequency (VLF), low frequency/high frequency (LF/HF), total power (TP), stress index (SI), standard deviation of the NN interval (SDNN), and the square root of the mean of the sum of the squares of differences between adjacent NN intervals (RMSSD).

14. The system of claim 9, wherein the data processing device is a computer, a notebook computer, a mobile phone, a console, or a personal digital assistant.

15. The system of claim 9, wherein the data processing device further comprises a data processing algorithm which calculates the customized essential oil blend based on the HRV parameters, a personal profile and the HRV-essential oil database.

16. The system of claim 15, wherein the personal profile comprises age, gender, race, medical history, allergic essential oils, preference of essential oils, and physical conditions.

17. The system of claim 9, wherein the customized essential oil blend comprises at least one of a pure essential oil and an existing essential oil blend collection.
